(19) United States
(12) Patent Application Publication
(10) Pub. No.: US 2013/0237548 A1
(43) Pub. Date: Sep. 12, 2013

(54) INJECTABLE EMULSION OF SEDATIVE HYPNOTIC AGENT

(71) Applicants: Helen Blade, Cheshire (GB); Stephen David Cosgrove, Cheshire (GB)

(72) Inventors: Helen Blade, Cheshire (GB); Stephen David Cosgrove, Cheshire (GB)

(73) Assignee: ASTRAZENECA AB, Sodertalje (SE)

(21) Appl. No.: 13/695,140

(22) PCT Filed: Apr. 21, 2011
(86) PCT No.: PCT/SE11/50489

§ 371 Date: May 23, 2013

Related U.S. Application Data
(60) Provisional application No. 61/329,662, filed on Apr. 30, 2010.

(51) Int. Cl. C07D 413/04

(52) U.S. Cl. CPC C07D 413/04
USPC 514/254.03; 544/367

(57) ABSTRACT

Polymorphs of 7-methyl-5-(3-piperazin-1-ylmethyl-[1,2,4]oxadiazol-5-yl)-2-(4-trifluoromethoxybenzyl)-2,3-dihydroisoindol-1-one mesylate salt, methods of making these polymorphs and uses thereof.

### FIELD OF THE INVENTION

The present invention relates to different crystal forms of 7-methyl-5-(3-piperazin-1-ylmethyl-[1,2,4]oxadiazol-5-yl)-2-(4-trifluoromethoxybenzyl)-2,3-dihydroisoindol-1-one mesylate salt, preparation thereof and uses thereof.

### BACKGROUND OF THE INVENTION

The metabotropic glutamate receptors (mGluR) are activated by glutamate, and have important roles in synaptic activity in the central nervous system, including neural plasticity, neural development and neurodegeneration.

Eight mGluR subtypes have been identified, which are divided into three groups based upon primary sequence similarity, signal transduction linkages, and pharmacological profile. Group-I includes mGluR1 and mGluR5, which activate phospholipase C and the generation of an intracellular calcium signal. Group-II (mGluR2 and mGluR3) and Group-III (mGluR4, mGluR6, mGluR7, and mGluR8) mGluRs mediate inhibition of adenylyl cyclase activity and decrease cyclic AMP levels.

Recent advances in the elucidation of the neurophysiological roles of mGluRs have established these receptors as promising drug targets in the therapy of acute and chronic neurological and psychiatric disorders and chronic and acute pain disorders. Because of the physiological and pathophysiological significance of the mGluRs, there is a need for new drugs and compounds that can modulate mGluR function.

### BRIEF DESCRIPTION OF THE DRAWINGS

FIG. 1 shows the XRPD results of Polymorph A.

FIG. 2 shows the melting characteristics of Polymorph A.

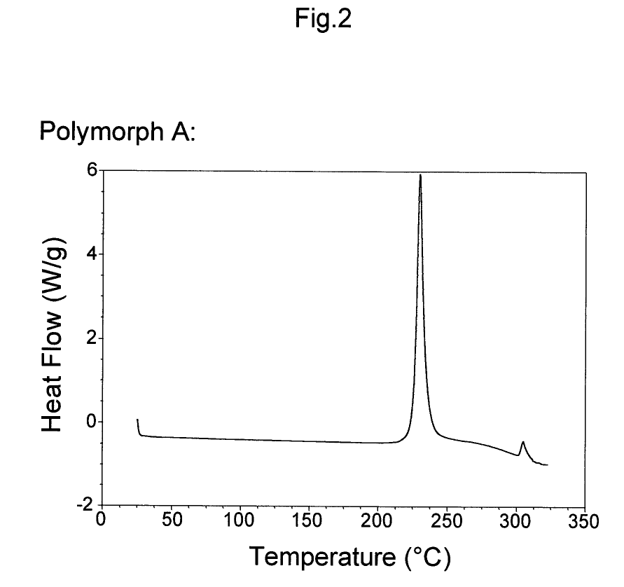

FIG. 3 shows the XRPD results of Polymorph C.

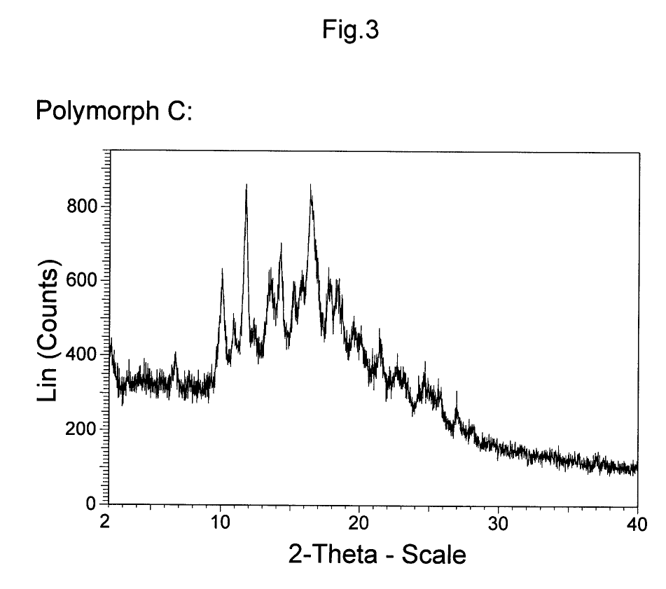

FIG. 4 shows the melting characteristics of Polymorph C.

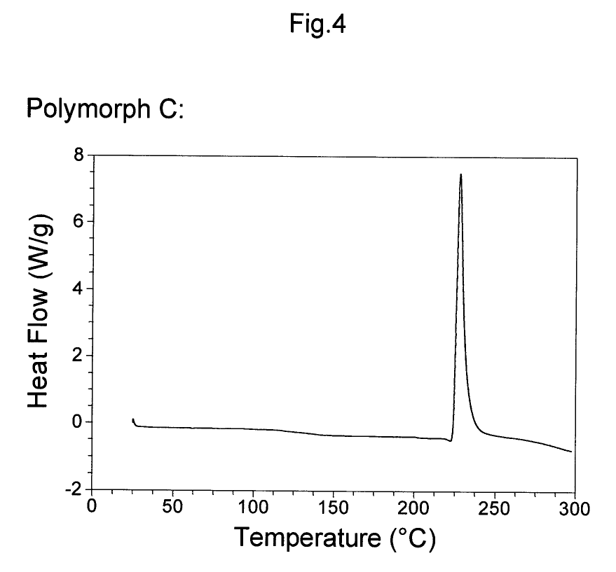

FIG. 5 shows the XRPD results of Polymorph D.

FIG. 6 shows the melting characteristics of Polymorph D.

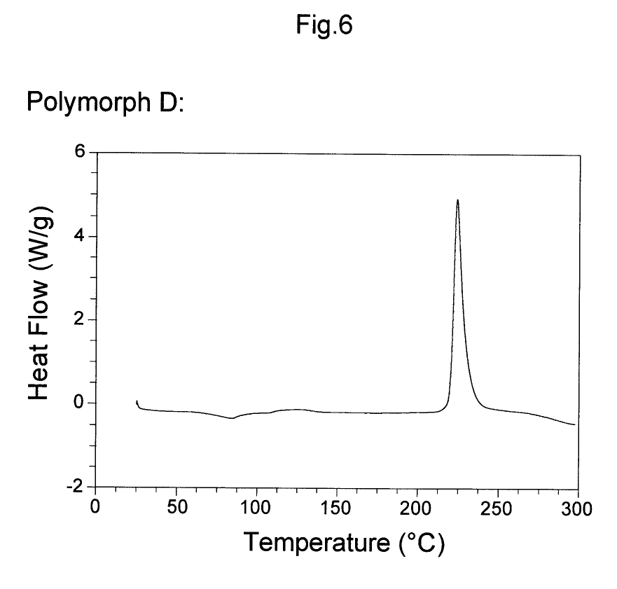

FIG. 7 shows the XRPD results of Polymorph E.

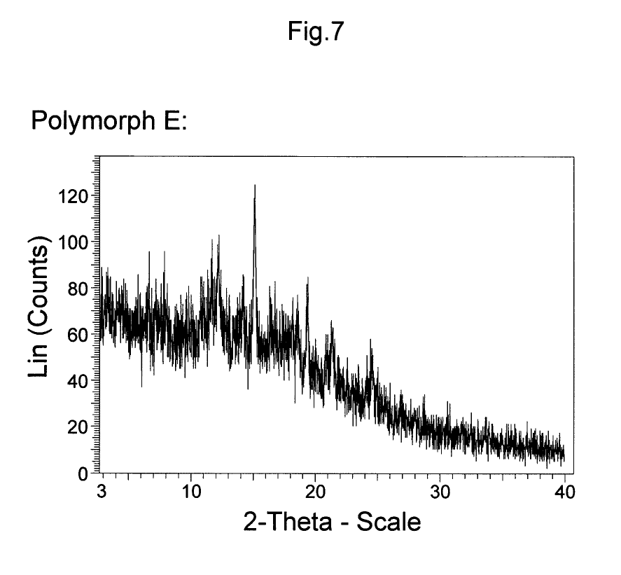

FIG. 8 shows the XRPD results of Polymorph F.

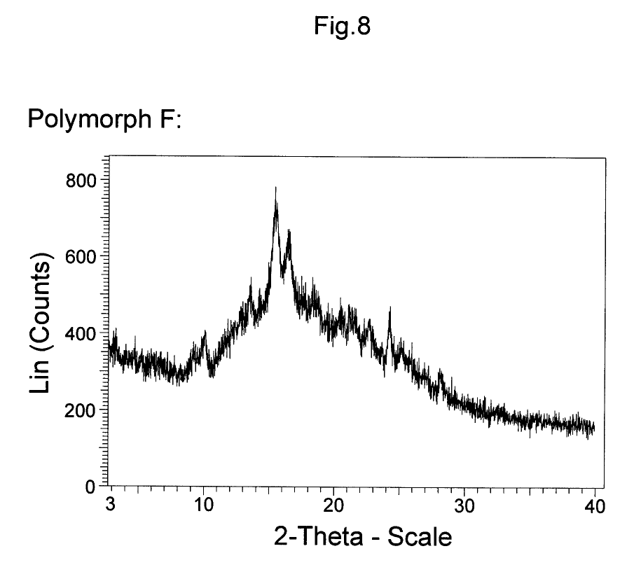

### DESCRIPTION OF THE EMBODIMENTS

7-Methyl-5-(3-piperazin-1-ylmethyl-[1,2,4]oxadiazol-5-yl)-2-(4-trifluoromethoxybenzyl)-2,3-dihydroisoindol-1-one may be prepared as described in U.S. Patent Application Publication No. 20080306077A1, which is incorporated by reference herein in its entirety. U.S. Patent Application Publication No. 20080306077A1 describes methods of making and using the aforesaid compound.

We have identified three different crystal forms of 7-methyl-5-(3-piperazin-1-ylmethyl-[1,2,4]oxadiazol-5-yl)-2-(4-trifluoromethoxybenzyl)-2,3-dihydroisoindol-1-one mesylate salt, identified herein as Polymorphs A, C and D.

We find that a first polymorph (described as “Polymorph A” in the Examples) of 7-methyl-5-(3-piperazin-1-ylmethyl-[1,2,4]oxadiazol-5-yl)-2-(4-trifluoromethoxybenzyl)-2,3-dihydroisoindol-1-one mesylate salt may be produced by a process described herein.

Therefore, one aspect of the invention provides a process of preparing Polymorph A of 7-methyl-5-(3-piperazin-1-ylmethyl-[1,2,4]oxadiazol-5-yl)-2-(4-trifluoromethoxybenzyl)-2,3-dihydroisoindol-1-one mesylate salt.

A further aspect of the invention provides a combination of two or more of the polymorphs described herein.

The polymorphs (e.g., Polymorphs A, C or D shown in the Examples) when in solution exhibit activity as modulators of metabotropic glutamate receptors and more particularly exhibit activity as potentiators of the mGluR2 receptor. It is contemplated that the compounds will be useful in therapy as pharmaceuticals, in particular, for the treatment of neurological and psychiatric disorders associated with glutamate dysfunction.

A further aspect of the invention is a method for the treatment of a subject suffering from any of the conditions discussed herein, whereby an effective amount of a polymorph as described herein is administered to a patient in need of such treatment.

Thus, the invention provides a polymorph as defined herein for use in therapy or the treatment of diseases mentioned herein.

The polymorphs defined herein are useful in therapy of neurological and psychiatric disorders in which the actions of metabotropic glutamate receptors, and particularly mGluR2 are involved including, but not limited to, disorders such as cerebral deficit subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, social phobia, obsessive compulsive disorder, and post-traumatic stress disorder (PTSD)), mood disorders (including depression, mania, bipolar disorders), circadian rhythm disorders (including jet lag and shift work), trigeminal neuralgia, hearing loss, tinnitus, macular degeneration of the eye, emesis, brain edema, pain (including acute and chronic pain states, severe pain, intractable pain, neuropathic pain, inflammatory pain, and post-traumatic pain), tardive dyskinesia, sleep disorders (including narcolepsy), attention deficit/hyperactivity disorder, and conduct disorder.

In use for therapy in a warm-blooded animal such as a human, a polymorph defined herein may be administered in the form of a pharmaceutical composition by any route including ingestion, administration by oral, intramuscular, subcutaneous, topical, intranasal, intraperitoneal, intrathoracical, intravenous, epidural, intrathecal, intracerebroventricular routes and by injection into the joints.

In a particular embodiment of the invention, the route of administration may be by ingestion or by an oral, intravenous or intramuscular route.

The dosage will depend on the route of administration, the severity of the disease, age and weight of the patient and other factors normally considered by an attending physician when determining the individual regimen and dosage level most appropriate for a particular patient.

For preparing pharmaceutical compositions from a polymorph defined herein, inert, pharmaceutically acceptable carriers can be either solid or liquid. Solid form preparations include powders, tablets, dispersible granules, capsules, cachets, and suppositories.

A solid carrier can be one or more substance, which may also act as a diluent, a flavoring agent, a solubilizer, a lubricant, a suspending agent, a binder, or a tablet-disintegrating agent; it can also be an encapsulating material.

In powders, the carrier is a finely divided solid, which is in a mixture with the finely divided polymorph. In tablets, the active component is mixed with a carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then placed in suitable sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Tablets, powders, cachets, and capsules can be used as solid dosage forms suitable for oral administration by ingestion.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (percent by weight), more preferably from 0.10 to 50% w, of the polymorph, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of a polymorph as defined herein for the manufacture of a medicament.

Also within the scope of the invention is the use of a polymorph defined herein for the manufacture of a medicament for the therapy of pain and/or anxiety.

Additionally provided is the use of a polymorph defined herein for the manufacture of a medicament for the therapy of conditions mentioned herein.

A further aspect of the invention is a method for therapy of a subject suffering from any of the conditions discussed above, whereby an effective amount of a polymorph defined herein is administered to a patient in need of such therapy.

Additionally, there is provided a pharmaceutical composition comprising at least one of a polymorph defined herein in association with a pharmaceutically acceptable carrier.

Particularly, there is provided a pharmaceutical composition comprising at least one of a polymorph defined herein in association with a pharmaceutically acceptable carrier for therapy, more particularly for therapy of the conditions mentioned herein.

Further, there is provided a pharmaceutical composition comprising at least one of a polymorph defined herein in association with a pharmaceutically acceptable carrier use in any of the conditions mentioned herein.

The term composition is also intended to include the formulation of the active component with encapsulating material as a carrier providing a capsule in which the active component (with or without other carriers) is surrounded by a carrier which is thus in association with it. Similarly, cachets are included.

As used herein, the term “therapy” also includes prophylactic therapy unless there are specific indications to the contrary. The term “therapeutic” and “therapeutically” should be construed accordingly. The term “therapy” within the context of the present invention encompasses administering an effective amount of a compound of the present invention, to mitigate either a pre-existing disease state, acute or chronic, or a recurring condition. This definition also encompasses prophylactic therapies for prevention of recurring conditions and continued therapy for chronic disorders. Similarly, “treatment” or “treating” when used herein includes the prophylactic administration of an effective amount of a compound of the present invention, to mitigate either a pre-existing disease state, acute or chronic, or a recurring condition.

“Ambient temperature” refers to a temperature between 25° C. and 30° C.;

“rel vol” refers to relative volumes;

“rel wt” refers to relative weight;

As will be appreciated by those of skill in the art, X-Ray powder diffraction (XRPD) patterns may be obtained and measured by a variety of instruments and methods. Similarly, thermal gravimetric analysis (TGA) and differential scanning calorimetry (DSC) may be carried out with a variety of instruments and methods. Further, processes described herein are exemplary processes and amounts, volumes, temperatures and other parameters may be varied as will be appreciated by those of skill in the art, while still achieving the desired result. Thus, the particular processes, methods and procedures described herein are not to be construed as limiting the invention in any way but are provided as exemplary processes, methods and procedures.

### Experimental Methods

### X-Ray Powder Diffraction (XRPD).

Powder X-ray diffraction patterns were recorded using two different diffractometers.

The X-ray powder diffractions pattern of Polymorph A was collected using a Bruker D5000 diffractometer was (wavelength of X-rays 1.5418 Å Cu source, Voltage 40 kV, filament emission 40 mA). Samples were scanned from 2-40° 2θ using a step size of 0.02° and a 1 second per step time count.

X-ray powder diffractions patterns of Polymorphs B-F were collected on a Bruker D8 diffractometer (wavelength of X-rays 1.5418 Å Cu source, Voltage 40 kV, filament emission 40 mA) with a humidity stage attached. XRPD patterns were recorded under varying humidity conditions; the material was scanned from 2-40° 2θ using a step size of 0.014° and a 0.2 seconds per step time count.

### Thermal Gravimetric Analysis (TGA):

TGA was recorded using a TA Instrument TGA, Q5000 series. Typically less than 5 mg of material, contained in a 100 μL platinum pan, was heated over the temperature range 25° C. to 325° C. at a constant heating rate of 10° C. per minute. A nitrogen purge gas was used with flow rate 100 mL per minute.

### Differential Scanning Calorimetry (DSC):

Differential scanning calorimetry was performed using a TA Instruments model Q1000. A sample (approximately 2 mg) was weighed into an aluminium sample pan and transferred to the DSC. The instrument was purged with nitrogen at 50 mL/min and data collected between 25° C. and 300° C., using a heating rate of 10° C./minute.

### EXAMPLES

The invention will further be described in more detail by the following Examples which describe methods whereby compounds of the present invention may be prepared, purified, analyzed and biologically tested, and which are not to be construed as limiting the invention.

### Example 1

### Preparation of Polymorph A of 7-methyl-5-(3-piperazin-1-ylmethyl-[1,2,4]oxadiazol-5-yl)-2-(4-trifluoromethoxybenzyl)-2,3-dihydroisoindol-1-one mesylate salt

Aqueous methanesulfonic acid is added to a warm solution of 4-{5-[7-methyl-1-oxo-2-(4-trifluoromethoxy-benzyl)-2,3-dihydro-1H-isoindol-5-yl]-[1,2,4]oxadiazol-3-ylmethyl}-piperazine-1-carboxylic acid tert-butyl ester in aqueous 1-butanol and the resulting solution held until complete reaction. The solution is screened and the solvent composition is altered by addition of further 1-butanol. Crystallization is achieved by seeding before a short series of controlled cooling—heating cycles. The resulting slurry is filtered and washed with 1-butanol. The solid is dried to constant weight in a vacuum oven overnight.

Particularly, 4-{5-[7-Methyl-1-oxo-2-(4-trifluoromethoxy-benzyl)-2,3-dihydro-1H-isoindol-5-yl]-[1,2,4]oxadiazol-3-ylmethyl}-piperazine-1-carboxylic acid tert-butyl ester (100% w/w, 1.0 mol eq) is charged to a vessel with 1-butanol (2.5 rel vol) and water (0.3 rel vol). The mixture is agitated and heated to a temperature in the range 85 to 90° C.

Methanesulfonic acid (70% w/w, 1.1 mol eq) is added via a dropping funnel over at least 5 min and washed in with water (0.1 rel vol) and the mixture stirred at a temperature in the range 85 to 90° C. for at least 18 h.

The vessel is cooled to a temperature in the range 65 to 70° C. and the contents are slowly transferred to a three liter jacketed crystallizer held at 69° C. with slow agitation and rinsed in with 1-butanol (2.0 rel vol).

The contents of the crystallizer are heated to a temperature in the range 82 to 88° C. and 1-butanol (8.0 rel vol) is added over at least 30 min while maintaining the contents >82° C.

The agitation rate in the crystallizer is increased, the temperature of the contents lowered to 77 to 78° C. and a seed of micronized 7-methyl-5-(3-piperazin-1-ylmethyl-[1,2,4]oxadiazol-5-yl)-2-(4-trifluoromethoxybenzyl)-2,3-dihydroisoindol-1-one mesylate salt (0.001 rel wt) added and the contents of the crystallizer held at a temperature in the range 75 to 78° C. for at least 30 min.

The crystallizer and contents are cooled to a temperature in the range 13 to 18° C. over at least 1.5 h and held at a temperature in the range 13 to 18° C. for at least 1 h.

The crystallizer and contents are warmed to a temperature in the range 65 to 70° C., held in that temperature range for 1 hour, then cooled to a temperature in the range 13 to 18° C. over at least 1.5 hours and held in that temperature range for at least 1 hour. The heating and cooling cycle is repeated once and the crystalline product recovered by filtration, fully de-liquored, washed with 1-butanol (2.0 rel vol), de-liquored again and dried under vacuum for at least 1 h at a temperature in the range 40 to 45° C. to constant weight. This was material was collected and analyzed by XRPD.

Polymorph A Primary peak values:

| 2-theta/° | | |
|---|---|---|
| (λ = 1.5418 Å) | d value | counts |
| 8.0 | 11.1 | 250 |
| 17.8 | 4.98 | 1932 |
| 18.4 | 4.81 | 841 |
| 19.5 | 4.55 | 1471 |
| 21.0 | 4.22 | 1841 |

Polymorph A Secondary peak values:

| 2-theta/° | | |
|---|---|---|
| (λ = 1.5418 Å) | d value | counts |
| 11.8 | 7.5 | 264 |
| 15.6 | 5.7 | 465 |
| 17.1 | 5.2 | 469 |
| 20.4 | 4.35 | 758 |
| 21.3 | 4.2 | 471 |

An identified Polymorph B is not described herein.

### Example 2

### Preparation of metastable Polymorph C of 7-methyl-5-(3-piperazin-1-ylmethyl-[1,2,4]oxadiazol-5-yl)-2-(4-trifluoromethoxybenzyl)-2,3-dihydroisoindol-1-one mesylate salt

A saturated solution of 7-methyl-5-(3-piperazin-1-ylmethyl-[1,2,4]oxadiazol-5-yl)-2-(4-trifluoromethoxybenzyl)-2,3-dihydroisoindol-1-one mesylate salt was made up in water, filtered and ˜1 mL was placed into a small vial. To this ˜2 mL of heptane was added rapidly to induce a rapid crystallization. The water and heptane formed an immiscible mixture that on evaporation under compressed air formed a white sticky material on the bottom of the vial. This was material was collected and analysed by XRPD.

Polymorph C Primary peak values:

| 2-theta/° | | |
|---|---|---|
| (λ = 1.5418 Å) | d value | counts |
| 10.0 | 8.8 | 622 |
| 15.2 | 5.8 | 591 |
| 16.4 | 5.4 | 825 |

Polymorph C Secondary peak values:

| 2-theta/° | | |
|---|---|---|
| (λ = 1.5418 Å) | d value | counts |
| 11.7 | 7.5 | 863 |
| 14.2 | 6.2 | 693 |

### Example 3

### Preparation of Polymorph D, a hemihydrate of 7-methyl-5-(3-piperazin-1-ylmethyl-[1,2,4]oxadiazol-5-yl)-2-(4-trifluoromethoxybenzyl)-2,3-dihydroisoindol-1-one mesylate salt

A saturated solution of 7-methyl-5-(3-piperazin-1-ylmethyl-[1,2,4]oxadiazol-5-yl)-2-(4-trifluoromethoxybenzyl)-2,3-dihydroisoindol-1-one mesylate salt was made up in water and then filtered, ˜1 mL was placed into a small vial. This was then placed into a vacuum oven set at room temperature and left to evaporate. A white precipitate formed during evaporation. This material was collected and analysed by XRPD.

Polymorph D Primary peak values:

| 2-theta/° | | |
|---|---|---|
| (λ = 1.5418 Å) | d value | counts |
| 10.9 | 8.1 | 977 |
| 15.8 | 5.6 | 766 |
| 17.7 | 5.0 | 1003 |
| 18.0 | 4.93 | 1056 |
| 20.2 | 4.39 | 913 |

Polymorph D Secondary peak values:

| 2-theta/° | | |
|---|---|---|
| (λ = 1.5418 Å) | d value | counts |
| 16.9 | 5.2 | 637 |
| 11.8 | 7.5 | 1668 |
| 14.3 | 6.2 | 1171 |

### In Situ Forms Observed:

### Example 4

### Preparation of Polymorph E, a hemihydrate of 7-methyl-5-(3-piperazin-1-ylmethyl-[1,2,4]oxadiazol-5-yl)-2-(4-trifluoromethoxybenzyl)-2,3-dihydroisoindol-1-one mesylate salt

A saturated solution of 7-methyl-5-(3-piperazin-1-ylmethyl-[1,2,4]oxadiazol-5-yl)-2-(4-trifluoromethoxybenzyl)-2,3-dihydroisoindol-1-one mesylate salt was made up in propan-2-ol, filtered and ˜1 mL was placed into a small vial covered with plastic film pierced with a small pinhole. The vial was placed at 5° C. and the solvent allowed to evaporate slowly to just before dryness. A white precipitate formed during this time, which was collected and analyzed when wet by XRPD.

### Example 5

### Preparation of Polymorph F, a hemihydrate of 7-methyl-5-(3-piperazin-1-ylmethyl-[1,2,4]oxadiazol-5-yl)-2-(4-trifluoromethoxybenzyl)-2,3-dihydroisoindol-1-one mesylate salt

A saturated solution of 7-methyl-5-(3-piperazin-1-ylmethyl-[1,2,4]oxadiazol-5-yl)-2-(4-trifluoromethoxybenzyl)-2,3-dihydroisoindol-1-one mesylate salt was made up in water, filtered and ˜1 mL was placed into a small vial. Then, using a suitable heating/cooling block, a cooling crystallization experiment was performed by heating the solution to 90° C. and then cooling back to room temperature at a cooling rate of 5° C./minute. On cooling to room temperature nothing was observed in the solution remaining, the solvent was evaporated off slowly at ambient temperature producing a white precipitate. This was collected and analyzed when wet by XRPD.

### DSC Details of the Isolated Forms:

Forms A, C and D exhibit an exothermic event in the DSC at 224-230° C. (onset 220-226° C.)

TGA analysis performed on Polymorph D indicated that the material was solvated. A weight loss of 1.3% was observed that indicates that this form may be a hemi-hydrate 1.5%.

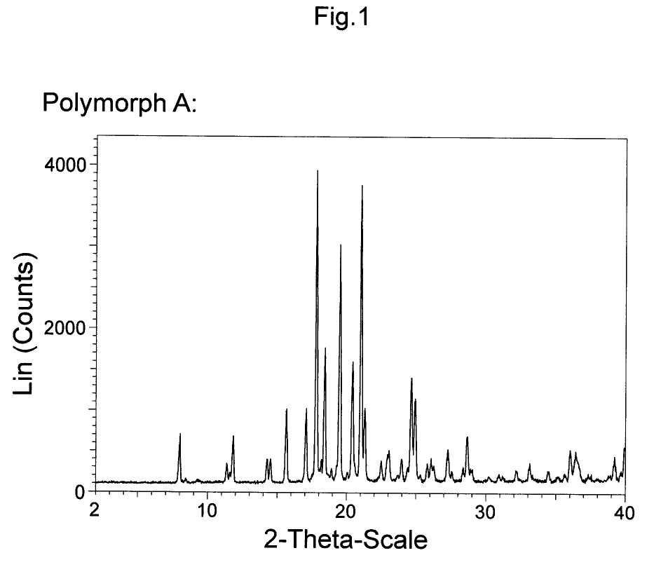

1. 7-Methyl-5-(3-piperazin-1-ylmethyl-[1,2,4]oxadiazol-5-yl)-2-(4-trifluoromethoxybenzyl)-2,3-dihydroisoindol-1-one mesylate salt Polymorph A, which has an XRPD pattern as shown in FIG. 1.

2. 7-Methyl-5-(3-piperazin-1-ylmethyl-[1,2,4]oxadiazol-5-yl)-2-(4-trifluoromethoxybenzyl)-2,3-dihydroisoindol-1-one mesylate salt, Polymorph A according to claim 1, wherein 2-theta, d values and counts as measured by XRPD are:

| 2-theta/° | | |
|---|---|---|
| (λ = 1.5418 Å) | d value | counts |
| 8.0 | 11.1 | 250 |
| 17.8 | 4.98 | 1932 |
| 18.4 | 4.81 | 841 |
| 19.5 | 4.55 | 1471 |
| 21.0 | 4.22 | 1841. |

3. 7-Methyl-5-(3-piperazin-1-ylmethyl-[1,2,4]oxadiazol-5-yl)-2-(4-trifluoromethoxybenzyl)-2,3-dihydroisoindol-1-one mesylate salt, Polymorph A, according to claim 2 wherein additional 2-theta, d values and counts as measured by XRPD are:

| 2-theta/° | | |
|---|---|---|
| (λ = 1.5418 Å) | d value | counts |
| 11.8 | 7.5 | 264 |
| 15.6 | 5.7 | 465 |
| 17.1 | 5.2 | 469 |
| 20.4 | 4.35 | 758 |
| 21.3 | 4.2 | 471. |

4. A pharmaceutical composition comprising 7-methyl-5-(3-piperazin-1-ylmethyl-[1,2,4]oxadiazol-5-yl)-2-(4-trifluoromethoxybenzyl)-2,3-dihydroisoindol-1-one mesylate salt, Polymorph A, according to claim 1, and at least one pharmaceutically acceptable carrier.

5. A method for the treatment of pain in a warm-blooded animal, comprising the step of administering to said animal in need of such therapy a therapeutically effective amount of 7-methyl-5-(3-piperazin-1-ylmethyl-[1,2,4]oxadiazol-5-yl)-2-(4-trifluoromethoxybenzyl)-2,3-dihydroisoindol-1-one mesylate salt, Polymorph A, according to claim 1.

6. A method for the treatment of anxiety in a warm-blooded animal, comprising the step of administering to said animal in need of such therapy a therapeutically effective amount of 7-methyl-5-(3-piperazin-1-ylmethyl-[1,2,4]oxadiazol-5-yl)-2-(4-trifluoromethoxybenzyl)-2,3-dihydroisoindol-1-one mesylate salt, Polymorph A, according to claim 1.

7. A method of making 7-methyl-5-(3-piperazin-1-ylmethyl-[1,2,4]oxadiazol-5-yl)-2-(4-trifluoromethoxybenzyl)-2,3-dihydroisoindol-1-one mesylate salt, Polymorph A according to claim 1, comprising:
dissolving 4-{5-[7-methyl-1-oxo-2-(4-trifluoromethoxy-benzyl)-2,3-dihydro-1H-isoindol-5-yl]-[1,2,4]oxadiazol-3-ylmethyl}-piperazine-1-carboxylic acid tert-butyl ester in 10% water/90% 1-butanol with agitation at 85 to 90° C.;
slowly adding methanesulfonic acid and stirring and maintaining the mixture at 85 to 90° C. for at least 18 h;
slowly diluting the mixture with 10 volumes of 1-butanol while maintaining the mixture at >82° C.;
with agitation, cooling the mixture to 77 to 78° C., adding a seed of micronised 7-methyl-5-(3-piperazin-1-ylmethyl-[1,2,4]oxadiazol-5-yl)-2-(4-trifluoromethoxybenzyl)-2,3-dihydroisoindol-1-one mesylate salt, and maintaining the temperature to 75 to 78° C. for at least 30 min;
slowly cooling the mixture to 13 to 18° C. over at least 1.5 h and maintaining at 13 to 18° C. for at least 1 h;
recovering the crystalline product.

8. 7-Methyl-5-(3-piperazin-1-ylmethyl-[1,2,4]oxadiazol-5-yl)-2-(4-trifluoromethoxybenzyl)-2,3-dihydroisoindol-1-one mesylate salt Polymorph A made by the method of claim 7.

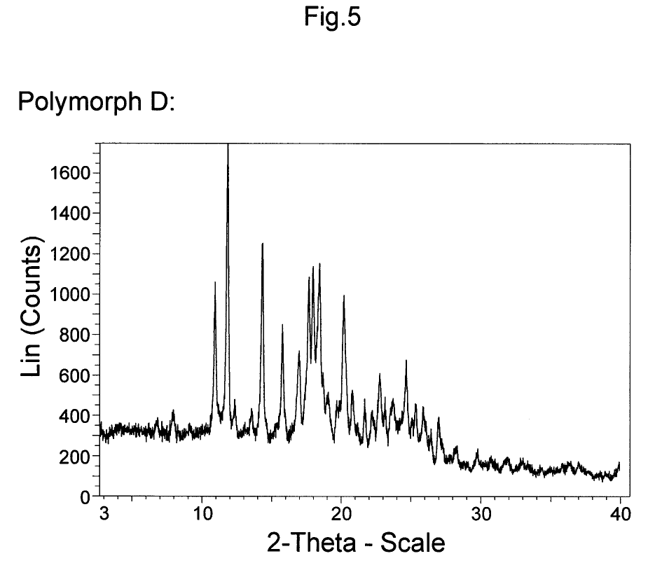

9. 7-Methyl-5-(3-piperazin-1-ylmethyl-[1,2,4]oxadiazol-5-yl)-2-(4-trifluoromethoxybenzyl)-2,3-dihydroisoindol-1-one mesylate salt Polymorph D, which has an XRPD pattern as shown in FIG. 5.

10. 7-Methyl-5-(3-piperazin-1-ylmethyl-[1,2,4]oxadiazol-5-yl)-2-(4-trifluoromethoxybenzyl)-2,3-dihydroisoindol-1-one mesylate salt, Polymorph D according to claim 9, wherein 2-theta, d values and counts as measured by XRPD are:

| 2-theta/° | | |
|---|---|---|
| (λ = 1.5418 Å) | d value | counts |
| 10.9 | 8.1 | 977 |
| 15.8 | 5.6 | 766 |
| 17.7 | 5.0 | 1003 |
| 18.0 | 4.93 | 1056 |
| 20.2 | 4.39 | 913 |

11. 7-Methyl-5-(3-piperazin-1-ylmethyl-[1,2,4]oxadiazol-5-yl)-2-(4-trifluoromethoxybenzyl)-2,3-dihydroisoindol-1-one mesylate salt, Polymorph D, according to claim 10 wherein additional 2-theta, d values and counts as measured by XRPD are:

| 2-theta/° | | |
|---|---|---|
| (λ = 1.5418 Å) | d value | counts |
| 16.9 | 5.2 | 637 |
| 11.8 | 7.5 | 1668 |
| 14.3 | 6.2 | 1171. |

12. A pharmaceutical composition comprising 7-methyl-5-(3-piperazin-1-ylmethyl-[1,2,4]oxadiazol-5-yl)-2-(4-trifluoromethoxybenzyl)-2,3-dihydroisoindol-1-one mesylate salt, Polymorph D, according to claim 9, and at least one pharmaceutically acceptable carrier.

13. A method for the treatment of pain in a warm-blooded animal, comprising the step of administering to said animal in need of such therapy a therapeutically effective amount of 7-methyl-5-(3-piperazin-1-ylmethyl-[1,2,4]oxadiazol-5-yl)-2-(4-trifluoromethoxybenzyl)-2,3-dihydroisoindol-1-one mesylate salt, Polymorph D, according to claim 9.

14. A method for the treatment of anxiety in a warm-blooded animal, comprising the step of administering to said animal in need of such therapy a therapeutically effective amount of 7-methyl-5-(3-piperazin-1-ylmethyl-[1,2,4]oxadiazol-5-yl)-2-(4-trifluoromethoxybenzyl)-2,3-dihydroisoindol-1-one mesylate salt, Polymorph D, according to claim 9.

15. A method of making 7-methyl-5-(3-piperazin-1-ylmethyl-[1,2,4]oxadiazol-5-yl)-2-(4-trifluoromethoxybenzyl)-2,3-dihydroisoindol-1-one mesylate salt, Polymorph D according to claim 9, comprising:
preparing a saturated solution of 7-methyl-5-(3-piperazin-1-ylmethyl-[1,2,4]oxadiazol-5-yl)-2-(4-trifluoromethoxybenzyl)-2,3-dihydroisoindol-1-one mesylate salt in water;
evaporating the water solution to dryness under vacuum at room temperature;
recovering the crystalline product.

16. 7-Methyl-5-(3-piperazin-1-ylmethyl-[1,2,4]oxadiazol-5-yl)-2-(4-trifluoromethoxybenzyl)-2,3-dihydroisoindol-1-one mesylate salt Polymorph D made by the method of claim 15.
